(19) United States
(12) Patent Application Publication
(10) Pub. No.: US 2022/0248831 A1
(43) Pub. Date: Aug. 11, 2022

(54) Tabletop electronic device for personal water intake tracking with an interactive mobile application

(71) Applicant: Ashish Sonawane, Livingston, NJ (US)

(72) Inventor: Ashish Sonawane, Livingston, NJ (US)

(21) Appl. No.: 17/248,847

(22) Filed: Feb. 10, 2021

(51) Int. Cl. A45F 3/20; A61B 5/00; G16H 20/30

(52) U.S. Cl. CPC A45F 3/20; G16H 20/30; A61B 5/7455; A61B 5/6887

(57) ABSTRACT

Personal water consumption tabletop electronic device with an interactive mobile app is disclosed. This invention automatically captures the exact personal water consumption. This tabletop device is less prone to breakage, water leakage, and electronic malfunction. Any water bottle, cup, or container works with this device.

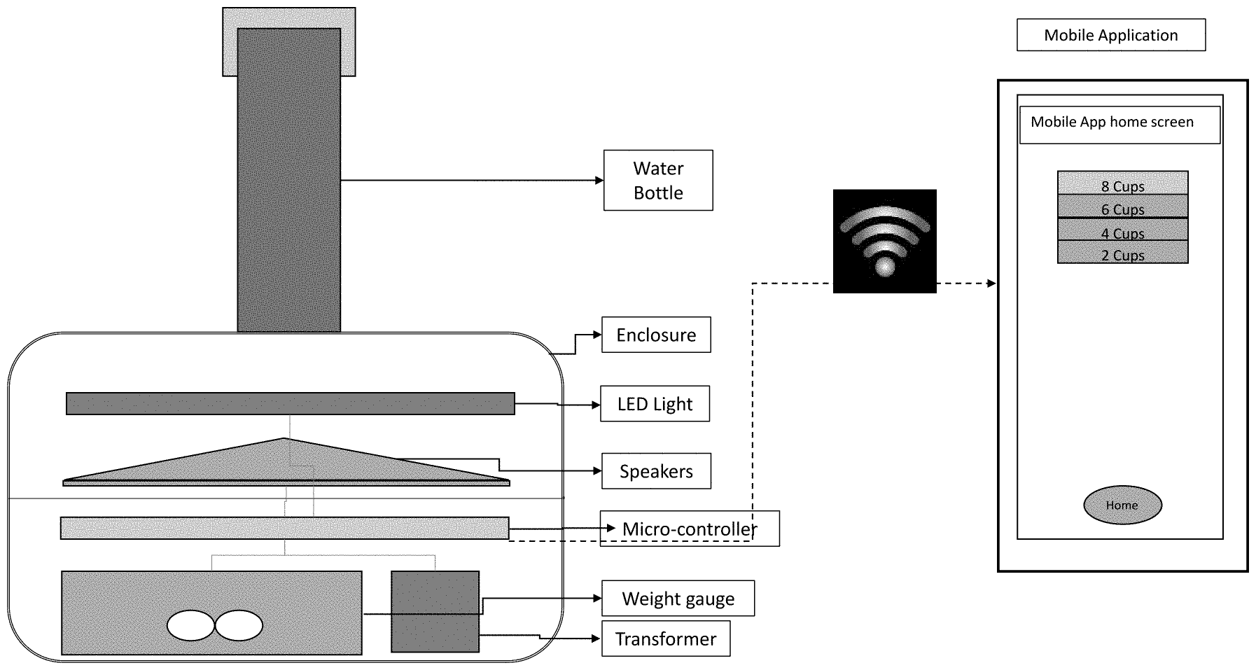

### CROSS REFERENCE TO RELATED APPLICATIONS

Provisional patent application No. 63/130,993

Filing Date: Dec. 28, 2020

Name of First Inventor: Ashish Sonawane

Title of Invention: Tabletop electronic device for personal water intake tracking with an interactive mobile app.

### BACKGROUND OF THE INVENTION

Problem Solved: In our day to day busy life, an adequate water drinking habit is difficult to build.

Existing water tracking devices are of 2 types. The first are mobile applications that allows user to add water consumed manually. And, the second types are attached to a specific water bottle. This doesn't allow to track water consumed from another bottle or a cup. These battery operated bottle devices have limited power to use sensors like motion, temperature sensors and audio output that limit interaction with user. Bottle devices are also prone to water leakages and breakages as it moves around with user. Also if you are not carrying the bottle device or misplaced it, you have to rely on manual data entry.

This invention tracks water consumption through bottles, cups or any other containers as long as the container fits on the top of the device.

### BRIEF SUMMARY OF THE INVENTION

This tabletop device collects the personal water consumption data automatically and sends it to a mobile application for tracking. It eliminates manual data entry errors from manual app., and bottle leakage & battery power limitations from water bottle mounted devices.

### DETAILED DESCRIPTION OF THE INVENTION

As stated above, in day to day busy life, an adequate water consumption is difficult to track. The invention claimed here solves this problem.

This tabletop device collects the personal water consumption data automatically and sends it to a mobile application for tracking.

The claimed invention differs from what currently exists. There is no electronic tabletop device that tracks and assists in building habit of adequate water consumption.

This invention is an improvement on what currently exists. This invention tracks adequate water consumption using any bottle or container as long as it fit on the top of the device.

Manual entry based mobile applications are prone to human error of missing entry, entering more or less than actual water consumed. The bottles devices have issues of water leakage, breakage, limited interactive features because of limited battery power.

This invention automatically captures the exact personal water consumption. This tabletop device is less prone to breakage, water leakage, and electronic malfunction.

Also, it can produce other devices like;

Automatic weighing scale

Exact volume-based beverage dispenser

The Version of the Invention Discussed Here Includes:
1. Wall mounted universal transformer
2. Wi-Fi/Bluetooth LE embedded memory microcontroller/microprocessor
3. Weighing sensor/Weight gauge
4. Speaker
5. Colored LED lights
6. Mobile application

### BRIEF DESCRIPTION OF DRAWING

In attached “drawing”, the Transformer (1) connects to the standard wall outlet. Mobile application (6) interacts with Wi-Fi/Bluetooth LE embedded microcontroller (2). Microcontroller (2) connect to weight gauge (3), Colored LED light (5), and the speaker (4). The water bottle is placed on the top of the enclosure.

### HOW THE INVENTION WORKS

The device is powered by plugging the transformer into the standard wall outlet. Mobile application sends instructions to microcontroller to calibrate the bottle or container. The User need to keep the water container/bottle on the device all the time during the usage. Water weight readings from the weight gauge are stored in microcontroller RAM and send back to the mobile app through Wi-Fi/Bluetooth. Water weight readings are then converted to water volume readings and displayed on the mobile application for tracking and reminders. Low water consumption rate triggers audio (speaker) and visual (color LED lights) stimulating signals through the microcontroller.

### HOW TO MAKE THE INVENTION

Create a user-friendly mobile application and connect it to a Wi-Fi Bluetooth microcontroller. Assemble input components—weight gauge and output components—Color LED light and speaker. Plugin the device, open the app and enjoy the invention.

All elements are necessary. Sensors to detect motion, heat, and light sensors would add functionality.

### HOW TO USE THE INVENTION

User can place any drinking water bottle on the top of the device during the usage. Real-time water consumption is tracked on mobile application.

Additionally: This invention can be used in an automatic rain gauge to measure accurate, real-time rainfall in inches.

The lights and sound simulation can be used for pet potty training & build sleeping habits at an early age.

Also, it can create:

Automatic weighing scale

Exact volume-based beverage dispenser

1. This invention claims that the tabletop electronic device uses a weighing sensor to measure the water consumed by the user from any container.
The device communicate this data using Wi-Fi and Bluetooth microcontroller or microprocessor to the mobile application for personal water intake tracking.
The invention claims to use water consumption data, visual stimulus (LED lights), and audio stimulus (speakers) from the device to build personal healthy water consumption habits.
